(19) United States
(12) Patent Application Publication
(10) Pub. No.: US 2025/0076168 A1
(43) Pub. Date: Mar. 6, 2025

(54) MODULAR APPARATUS FOR ACCELERATED MECHANICAL TESTING OF IMPLANTABLE LEADS

(71) Applicant: BOARD OF TRUSTEES OF THE UNIVERSITY OF ARKANSAS, Little Rock, AR (US)

(72) Inventors: Anil Thota, Plymouth, MN (US); Sathyakumar Kuntaegowdanahalli, Bentonville, AR (US); Andres Pena, St. Paul, MN (US)

(73) Assignee: BOARD OF TRUSTEES OF THE UNIVERSITY OF ARKANSAS, Little Rock, AR (US)

(21) Appl. No.: 18/821,056

(22) Filed: Aug. 30, 2024

Related U.S. Application Data
(60) Provisional application No. 63/535,510, filed on Aug. 30, 2023.

(51) Int. Cl. G01N 3/06; A61N 1/05

(52) U.S. Cl. CPC G01N 3/066; G01N 3/068; A61N 1/0551; G01N 2203/0226

(57) ABSTRACT

Embodiments pertain to a system for testing the mechanical stability of a material. Such systems generally include: (1) a chamber with a first end and a second end that are on opposite ends of the chamber; (2) a motion actuating unit associated with the first end of the chamber; (3) a first base area associated with motion actuating unit at or near the first end of the chamber; and (4) a second base area associated with the second end of the chamber. Additional embodiments pertain to methods for testing the mechanical stability of a material through the utilization of the systems.

### CROSS-REFERENCE TO RELATED APPLICATIONS

This application claims priority to U.S. Provisional Patent Application No. 63/535,510, filed on Aug. 30, 2023. The entirety of the aforementioned application is incorporated herein by reference.

### BACKGROUND

A need exists for more effective systems and methods for testing the mechanical stability of various materials, such as implantable electrodes. Numerous embodiments of the present disclosure aim to address the aforementioned need.

### SUMMARY

In some embodiments, the present disclosure pertains to a system for testing the mechanical stability of a material. In some embodiments, the systems of the present disclosure include: (1) a chamber with a first end and a second end that are on opposite ends of the chamber; (2) a motion actuating unit associated with the first end of the chamber; (3) a first base area associated with motion actuating unit at or near the first end of the chamber; and (4) a second base area associated with the second end of the chamber.

Additional embodiments of the present disclosure pertain to methods for testing the mechanical stability of a material through the utilization of the systems of the present disclosure. In some embodiments, the methods of the present disclosure include: (1) connecting a first end of a material to a first base area of the system; (2) connecting a second end of the material to a second base area of the system; (3) actuating the system's motion actuating unit, where the actuating moves the first base area and the immobilized material; and (4) measuring the mechanical stability of the material.

### BRIEF DESCRIPTION OF THE DRAWINGS

A better understanding of the present invention can be obtained when the following detailed description is considered in conjunction with the following drawings, in which:

FIG. 1 provides an illustration of a system for testing the mechanical stability of a material in accordance with various embodiments of the present disclosure. In this example, a motion actuating unit is positioned at or near the bottom of the system's chamber.

FIG. 2 provides an illustration of another system for testing the mechanical stability of a material in accordance with various embodiments of the present disclosure. In this example, a motion actuating unit is positioned at or near the top of the system's chamber.

FIG. 3 provides an illustration of another system for testing the mechanical stability of a material in accordance with various embodiments of the present disclosure. In this example, a motion actuating unit is also positioned at or near the top of the system's chamber. Furthermore, the system is operable to test the mechanical stability of multiple materials.

FIG. 4 provides a more detailed illustration of the motion actuating unit of the system illustrated in FIG. 3.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components that includes one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Current methods and systems for testing the mechanical stability of various materials, such as implantable electrodes, have numerous limitations. In particular, implantable electrical stimulation systems, such as peripheral nerve interfaces used for neurostimulation therapies that treat conditions like Parkinson's disease and chronic pain, consist of implantable leads that are inserted into the body and connected to a stimulator device. These implantable leads are subjected to a range of mechanical stresses during use, including repetitive strain, stress, and torsion. Moreover, it is important to ensure that such implantable leads can withstand these stresses without failing.

Peripheral nerve interfaces are devices that enable the flow of information between the peripheral nervous system and a computing machine. These interfaces have the potential to restore function after spinal cord injury, peripheral nerve injury, or limb loss. Peripheral nerve interfaces can also be used in autonomic control.

Peripheral nerve interfaces consist of electrodes that interface with the nervous system. The interfaces are generally connected via an electrode lead system to a stimulator or recorder electronics unit. There are several types of electrodes used for these interfaces. Moreover, lead systems connecting the electrodes to the stimulating and recording devices can vary. In some cases, the lead system consists of bundled and coiled wires enclosed in an external sheath, similar to those used in central nervous system (CNS) stimulation systems, such as spinal cord or deep brain stimulators.

For many clinical applications, targeting the peripheral nervous system can have advantages over targeting the central nervous system. Such advantages include reduced implantation risks and access to simpler, better-understood neural codes. However, targeting the nerves in the periphery can also present challenges, such as the potential for the nerves to slide and stretch during routine activities. This movement can compromise the stability of the electrode-nerve interface and the mechanical fatigue resistance of the leads, especially in systems where the electronics are housed at a distance from the electrode-nerve interface.

Mechanical fatigue that causes lead breakage or cracks in the insulation or protective sheath around the electrode lead wires can reduce system functionality, increase risk, and/or constitute device failure. Lead systems used in devices such as functional neuromuscular stimulators, spinal cord stimulators, and cardiac pacemakers have been extensively tested in-vitro and in-vivo to characterize their long-term survival under similar conditions. However, there is a need for a testing system that can specifically address the mechanical fatigue resistance of lead systems used in peripheral nerve interfaces, particularly those used in applications where the electronics are housed remotely from the electrode-nerve interface.

One way to test the mechanical reliability of implantable leads is through accelerated mechanical fatigue testing, which involves subjecting representative test specimens to repetitive strains that are representative of the types of stresses they will experience in use. This type of testing can be used to identify potential failure points and design improvements that can increase the durability and reliability of the leads.

However, there are currently limited options for performing mechanical fatigue testing on implantable leads. For instance, available equipment for such tests is often expensive and not well-suited for testing specific types of leads used in implantable electrical stimulation systems. Such limitations can make it difficult for manufacturers and researchers to accurately assess the mechanical reliability of these leads and make necessary improvements.

As such, a need exists for more effective systems and methods for testing the mechanical stability of various materials, such as implantable electrodes. Numerous embodiments of the present disclosure aim to address the aforementioned need.

### Systems for Testing the Mechanical Stability of Materials

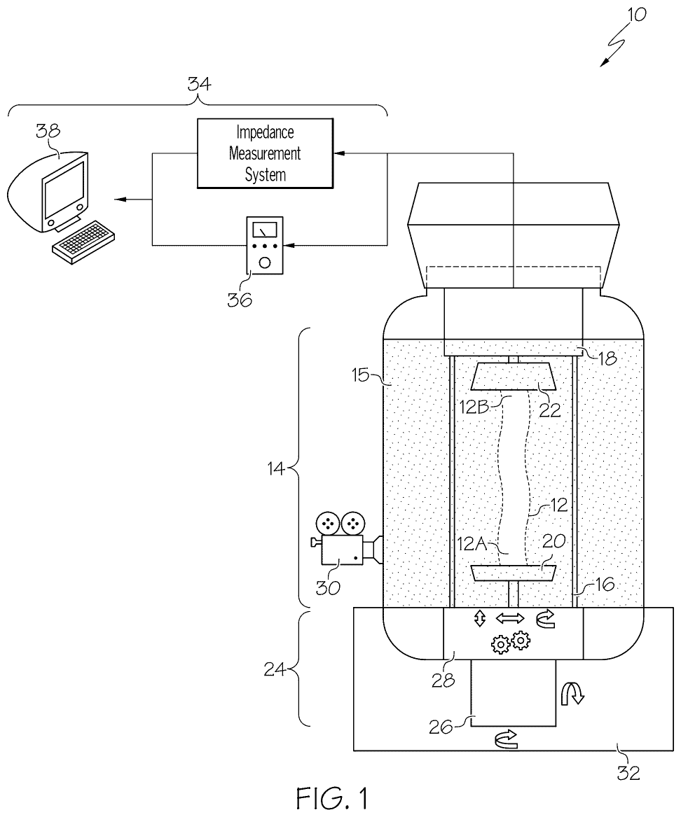

In some embodiments, the present disclosure pertains to a system for testing the mechanical stability of a material. For illustrative purposes, the systems of the present disclosure can be described with reference to FIG. 1, which illustrates a system 10 for testing the mechanical stability of a material 12. System 10 generally includes a chamber 14 with a first end 16 and a second end 18, which are on opposite ends of the chamber. System 10 also includes a motion actuating unit 24 associated with the first end 16 of the chamber. Additionally, system 10 includes a first base area 20 associated with motion actuating unit 24 at or near first end 16 of chamber 14, and a second base area 22 associated with the second end 18 of chamber 14.

First base area 20 is generally operational to connect to a first end 12A of material 12. Likewise, second base area 22 is operational to connect to a second end 12B of material 12. In some embodiments, first end 12A and second end 12B are on opposite ends of material 12. Additionally, motion actuating unit 24 is generally operational to move first base area 20 and immobilized material 12.

In some embodiments, motion actuating unit 24 includes a motor 26 operational for actuating motion. In some embodiments, motion actuating unit 24 further includes a motion transfer assembly 28 operational for transferring the actuated motion from motor 26 to first base area 20 for moving first base area 20 and immobilized material 12.

In some embodiments, system 10 also includes an optical monitoring system 30 operational to optically monitor material 12 associated with the system. In some embodiments, system 10 further includes a heater 32 associated with chamber 14 and operational to heat the chamber.

In some embodiments, system 10 also includes an impedance measurement system 34 operational to measure the impedance of material 12 associated with the system. In some embodiments, impedance measurement system 34 includes a multimeter 36. In some embodiments, impedance measurement system 34 also includes a computing device 38.

The systems of the present disclosure can have various configurations. For instance, in some embodiments illustrated in FIG. 1, system 10 includes a motion actuating unit 24 that is positioned at or near the bottom of chamber 14.

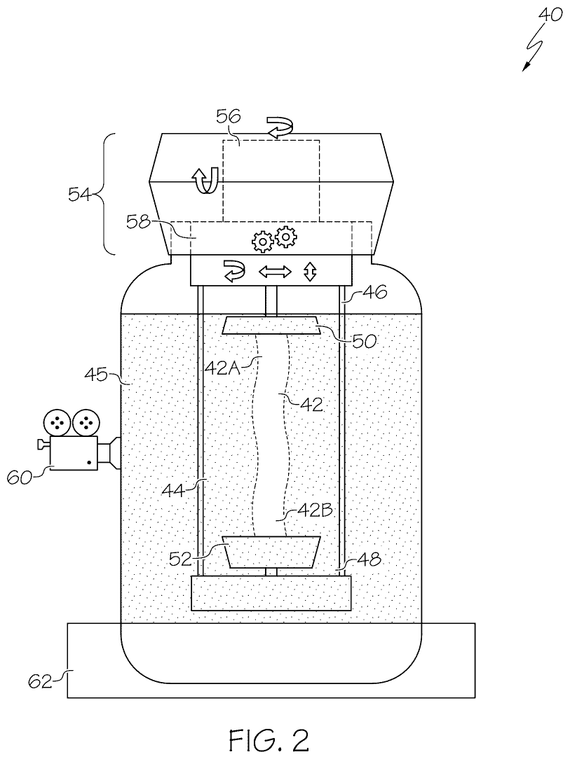

An alternative embodiment of a system of the present disclosure is shown in FIG. 2 as system 40. In this embodiment, system 40 includes a motion actuating unit 54 that is positioned at or near the top of chamber 44. In particular, system 40 is configured to test the mechanical stability of a material 42 and generally includes a chamber 44 with a first end 46 and a second end 48 that are on opposite ends of the chamber. Additionally, system 40 includes a first base area 50 associated with motion actuating unit 54 at or near first end 46 of chamber 44, and a second base area 52 associated with second end 48 of chamber 44.

First base area 50 is operational to connect to a first end 42A of material 42. Likewise, second base area 52 is operational to connect to a second end 42B of material 42. In some embodiments, first end 42A and second end 42B are on opposite ends of material 42. Additionally, motion actuating unit 54 is operational to move first base area 50 and immobilized material 42.

In some embodiments, motion actuating unit 54 includes a motor 56 operational for actuating motion. In some embodiments, motion actuating unit 54 further includes a motion transfer assembly 58 operational for transferring the actuated motion from motor 56 to first base area 50 for moving first base area 50 and immobilized material 42.

In some embodiments, system 40 also includes an optical monitoring system 60 operational to optically monitor material 42 associated with the system. In some embodiments, system 40 further includes a heater 62 associated with chamber 44 and operational to heat the chamber.

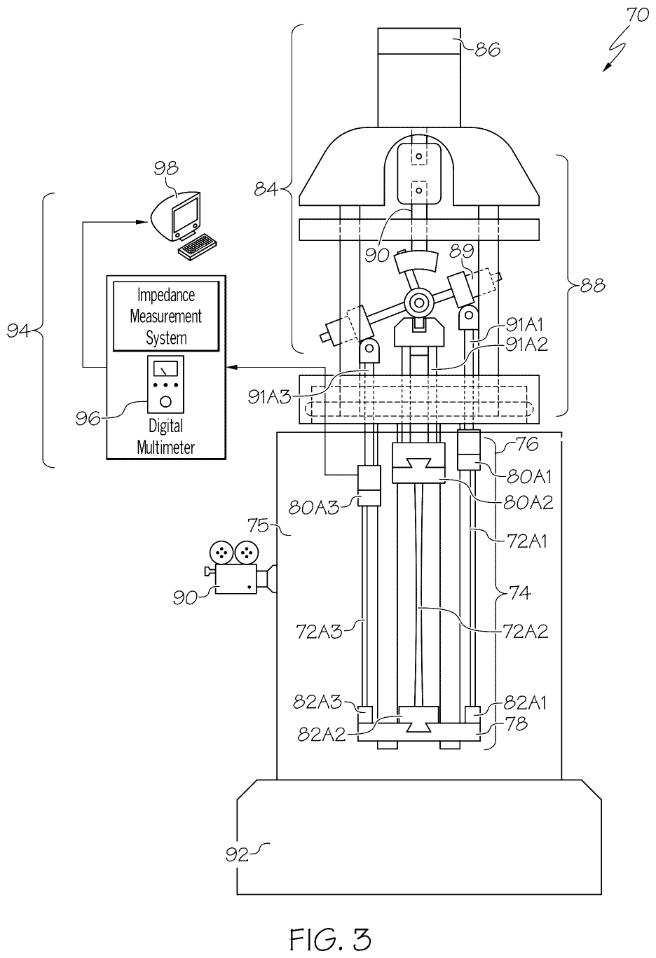

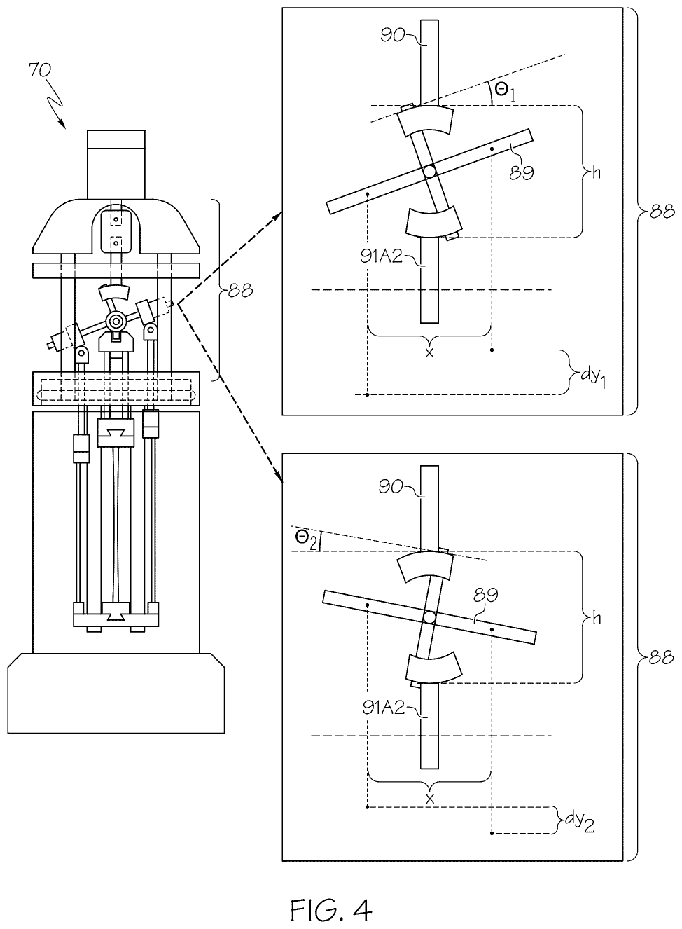

In some embodiments, the systems of the present disclosure may be operational for testing the mechanical stability of a plurality of materials. An example of such a system is illustrated in FIGS. 3-4 as system 70, which is operational for testing the mechanical stability of a plurality of materials 72A1, 72A2, and 72A3. In this example, system 70 generally includes a chamber 74 with a first end 76 and a second end 78, which are on opposite ends of the chamber. System 70 also includes a motion actuating unit 84 associated with the first end 76 of the chamber.

Additionally, system 70 includes a plurality of first base areas 80A1, 80A2, and 80A3 associated with motion actuating unit 84. Each of the first base areas 80A1, 80A2, and 80A3 are operational to connect to a first end of one of the plurality of materials 72A1, 72A1, and 72A3. Additionally, motion actuating unit 84 is operational to move each of the first base areas 80A1, 80A2, and 80A3 and each of the plurality of the immobilized materials 72A1, 72A2, and 72A3.

System 70 also includes a plurality of second base areas 82A1, 82A2, and 82A3. Each of the second base areas is associated with second end 78 of chamber 74. Additionally, each of the second base areas 82A1, 82A2 and 82A3 is operational to connect to a second end of one of the plurality of materials 72A1, 72A2, and 72A3.

Motion actuating unit 84 is generally operational to move each of the first base areas 80A1, 80A2, and 80A3 and each of the immobilized materials 72A1, 72A2, and 72A3. In some embodiments, the motion actuating unit 84 includes a motor 86 operational for actuating motion. In some embodiments, motion actuating 84 unit further includes a motion transfer assembly 88 operational for transferring the actuated motion from motor 86 to each of the first base areas 80A1, 80A2, and 80A3 for moving each of the first base areas and each of the immobilized materials 72A1, 72A2, and 72A3.

In some embodiments, motion transfer assembly 88 includes: a first vertical arm 90 connected to motor 86, a horizontal arm 89 connected to first vertical arm 90; and a plurality of second vertical arms 91A1, 91A2, and 91A3. In some embodiments, each of the plurality of second vertical arms is connected to horizontal arm 89 and one of the plurality of first base areas 80A1, 80A2, and 80A3.

In some embodiments, horizontal arm 89 is operational to transform motion from motor 86 to linear motion through an adjustable tilted axis. In some embodiments, the angle of the adjustable tilted axis can be adjusted to quickly change the amplitude of an applied strain. In some embodiments, each of the second vertical arms 91A1, 91A2, and 91A3 are operational to receive the transformed linear motion and move each of the first base areas 80A1, 80A2, and 80A3 and each of the plurality of the immobilized materials 72A1, 72A2, and 72A3.

In some embodiments, system 70 also includes an optical monitoring system 90 operational to optically monitor materials 72A1-72A3 associated with the system. In some embodiments, system 70 further includes a heater 92 associated with chamber 14 and operational to heat the chamber.

In some embodiments, system 70 also includes an impedance measurement system 94 operational to measure the impedance of materials 72A1-72A3 associated with the system. In some embodiments, impedance measurement system 94 includes a multimeter 96. In some embodiments, impedance measurement system 94 also includes a computing device 98.

As set forth in more detail herein, the systems of the present disclosure can include numerous components and configurations.

### Motion Actuating Units

The systems of the present disclosure can include various motion actuating units. For instance, in some embodiments, the motion actuating units of the present disclosure include a motor operational for actuating motion (e.g., motor 26, 56, and/or 86 shown in FIGS. 1, 2 and 3-4, respectively). In some embodiments, the actuated motion includes repetitive rotational motion. In some embodiments, the motor includes a speed-controlled motor. In some embodiments, the speed-controlled motor is in an axial configuration. In some embodiments, the speed-controlled motor is in a transverse configuration.

In some embodiments, the motor includes a DC motor. In some embodiments, the DC motor is a speed-controlled brushless DC motor that generates repetitive rotational motion.

In some embodiments, the motion actuating unit further includes a motion transfer assembly (e.g., motion transfer assembly 28, 58 and/or 88 shown in FIGS. 1, 2 and 3-4, respectively). In some embodiments, the motion transfer assembly is operational for transferring actuated motion from a motor to one or more first base areas for moving the one or more first base areas and one or more of the immobilized materials.

In some embodiments, the motion transfer assemblies of the present disclosure are operational to transform actuated motion from a motor into a linear motion. In some embodiments, the motion transfer assemblies of the present disclosure are operational to transform actuated motion from a motor into a strain profile of a material.

The motion actuating units of the present disclosure may be associated with the chambers of the present disclosure in various manners. For instance, in some embodiments, the motion actuating units of the present disclosure are coupled to a first end of a chamber through a sealing gasket.

### First and Second Base Areas

The systems of the present disclosure can include various first and second base areas. For instance, in some embodiments, each of the first and second base areas is in the form of a mounting structure that holds the materials of the present disclosure. In some embodiments, the first base areas of the present disclosure can include a moving clamp (e.g., moving clamps 20, 50 and/or 80A1-80A3 shown in FIGS. 1, 2, and 3-4, respectively). In some embodiments, the second base area includes a fixed clamp (e.g., fixed clamps 22, 52, and/or 82A1-82A3 shown in FIGS. 1, 2 and 3-4, respectively).

### Optical Monitoring Systems

In some embodiments, the systems of the present disclosure also include an optical monitoring system (e.g., optical monitoring systems 30, 60, and/or 90 shown in FIGS. 1, 2 and 3-4, respectively). In some embodiments, the optical monitoring system is operational to optically monitor the material associated with the system.

In some embodiments, the optical monitoring system includes a high-definition camera. In some embodiments, the optical monitoring system is operable to optically monitor the materials through videos, photos, or combinations thereof. In some embodiments, the optical monitoring system is associated with a chamber of the present disclosure.

### Heaters

In some embodiments, the systems of the present disclosure also include a heater (e.g., heater 32, 62, and/or 92 shown in FIGS. 1, 2 and 3-4, respectively). In some embodiments, the heater also includes a temperature controller module.

In some embodiments, the heaters of the present disclosure are associated with a chamber and operational to heat the chamber. In some embodiments, the heaters of the present disclosure are positioned at or near a first end of a chamber (e.g., heater 32 at first end 16 of chamber 14, as shown in FIG. 1). In some embodiments, the heaters of the present disclosure are positioned at or near a second end of a chamber (e.g., heater 62 at or near second end 48 of chamber 44, and/or heater 92 at or near second end 78 of chamber 44, as shown in FIGS. 2 and 3, respectively).

### Impedance Measurement Systems

In some embodiments, the systems of the present disclosure also include an impedance measurement system (e.g., impedance measurement system 34 and/or 94 shown in FIGS. 1 and 3, respectively). In some embodiments, the impedance measurement system is operational to measure the impedance of a material associated with a system. In some embodiments, the impedance measurement system includes a multimeter (e.g., multimeter 36 and/or 96 shown in FIGS. 1 and 3, respectively). In some embodiments, the impedance measurement system further includes a computing device (e.g., computing device 38 and/or 98 shown in FIGS. 1 and 3, respectively). In some embodiments, the impedance measurement system includes an electrochemical impedance spectrometer (EIS).

### Chambers

The systems of the present disclosure can include various chambers (chamber 14, 44 and/or 74 shown in FIGS. 1, 2 and 3, respectively). For instance, in some embodiments, the chamber is in the form of a cylinder. In some embodiments, the chamber is transparent. In some embodiments, the chamber is made of transparent materials, such as glass.

In some embodiments, the chamber is in the form of a vial. In some embodiments, the chamber is capable of being filled with a liquid (e.g., liquid 15, 45 and/or 75, as shown in FIGS. 1, 2, and 3, respectively). In some embodiments, the liquid includes saline solution.

In some embodiments, a chamber of the present disclosure may be filled with a saline solution maintained at physiologically relevant temperatures to simulate physiological conditions. In some embodiments, the saline solution may allow for the monitoring of material impedance and continuity values for continuous evaluation of a material's electrical and mechanical integrity. In some embodiments, the temperature of the saline solution can be controlled using a heater.

### System Configurations

The systems of the present disclosure can include various configurations. For instance, in some embodiments, a chamber can be installed in a vertical configuration, with motion actuating unit at or near the top or bottom, depending on the specific test requirements. As illustrated in FIG. 2, when a motion actuating unit 54 is installed at or near the top of a chamber, it actuates first base area 50 at or near the top of the chamber while the first base area 52 at or near the bottom of the chamber remains fixed. In some embodiments, the bottom opening of the chamber may be fitted with a sealing gasket and coupled to the system base, which houses components such as heater 62. This configuration is advantageous for testing scenarios where the material needs to be frequently removed or transferred (e.g., microscopy inspection), as it does not require the drainage of the saline solution, thereby allowing the specimen to be easily removed from the top.

Alternatively, in some embodiments illustrated in FIG. 1, motion actuating unit 24 can be installed at or near the bottom opening of a chamber, resulting in an inverted configuration where first base area 20 is at or near the bottom, and second base area 22 is at or near the top. In this embodiment, motion actuating unit 24 is housed within the system base along with heater 32. In some embodiments, this configuration allows access to the materials from the top opening of the chamber, thereby facilitating continuous impedance and continuity monitoring. Any connectors required to interface with materials being tested can be mounted to the fixed second base area 22 and kept above the level of saline solution 15.

### Methods for Testing the Mechanical Stability of a Material

Additional embodiments of the present disclosure pertain to methods for testing the mechanical stability of a material through the utilization of the systems of the present disclosure. With reference again to system 10 in FIG. 1 for illustrative purposes, the methods of the present disclosure include: (1) connecting a first end 12A of material 12 to a first base area 20 of the system; (2) connecting a second end 12B of material 12 to a second base area 22 of the system; (3) actuating motion actuating unit 24, where the actuating moves first base area 20 and immobilized material 12; and (4) measuring the mechanical stability of material 12. As set forth in more detail herein, the methods of the present disclosure can include numerous embodiments.

### Actuating of the Motion Actuating Unit

Various methods may be utilized to actuate motion actuating units. For instance, in some embodiments where the motion actuating unit includes a motor 26, the actuating includes actuating the motor. In some embodiments, the actuating results in repetitive rotational motion.

In some embodiments where the motion actuating unit further includes a motion transfer assembly 28, the actuating results in the transfer of the actuated motion from motor 26 to a first base area 20 for moving the first base area and the immobilized material 12. In some embodiments, motion transfer assembly 28 transforms the actuated motion from motor 26 into a linear motion. In some embodiments, motion transfer assembly 28 transforms the actuated motion from motor 26 into a strain profile of material 12.

### Optical Monitoring

In some embodiments, the methods of the present disclosure also include a step of optically monitoring a material 12 associated with system 12. In some embodiments, the optical monitoring occurs through the utilization of an optical monitoring system 30 associated with system 10.

### Heating.

In some embodiments, the methods of the present disclosure also include a step of heating chamber 14. In some embodiments, heating occurs through the actuation of a heater 32 associated with chamber 14.

### Tested Mechanical Stability

The methods and systems of the present disclosure may be utilized to test various mechanical stabilities of materials. For instance, in some embodiments, the tested mechanical stability includes, without limitation, strain, angular strain, linear strain, torsional strain, stress, torsion, mechanical fatigue, or combinations thereof. In some embodiments, the systems and methods of the present disclosure may be utilized to apply repetitive strains to potential failure points on the materials, thereby evaluating the durability and reliability of the materials (e.g., implantable leads).

In some embodiments, the testing of the mechanical stability of a material includes measuring the impedance of the material. In some embodiments, the impedance of the material is measured by an impedance measurement system 34, which may also include a multimeter 36 and a computing device 38.

Filling a Chamber with a Liquid

In some embodiments, the methods of the present disclosure also include a step of filling a chamber 14 with a liquid (e.g., liquid 15). In some embodiments, the liquid includes saline solution. In some embodiments, the filling occurs prior to measuring the mechanical stability of a material.

### Testing of Multiple Materials

In some embodiments, the methods of the present disclosure may be utilized to test the mechanical stability of multiple materials. In some embodiments, such methods may utilize systems of the present disclosure that are capable of immobilizing multiple materials, such as system 70 illustrated in FIGS. 3-4. With reference again to system 70 in FIGS. 3-4 for illustrative purposes, the methods of the present disclosure include: connecting each of the first base areas 80A1-A3 to a first end of one of the plurality of materials 72A1-A3; connecting each of the second base areas 82A1-82A3 to a second end of one of the plurality of the materials 72A1-72A3; actuating motion actuating unit 84, where the actuating moves each of the first base areas 80A1-A3 and each of the immobilized materials 72A1-A3; and measuring the mechanical stability of each of the materials.

In some embodiments, after actuation of motion actuating unit 84, horizontal arm 89 may transform motions from motor 89 to linear motion through an adjustable tilted axis. Thereafter, each of the second vertical arms 91A1-91A3 receive the transformed linear motion and move each of the first base areas 80A1-A3 and each of the plurality of the immobilized materials 72A1-A3.

### Materials

The methods and systems of the present disclosure may be utilized to test various materials. In some embodiments, the material includes an electrode. In some embodiments, the electrode includes an implantable electrode. In some embodiments, the electrode includes an implantable electrode lead system. In some embodiments, the electrode includes an implantable electrode lead system used in peripheral nerve interfaces.

### Advantages and Applications

The systems and methods of the present disclosure can provide numerous advantages and applications. In particular, the systems and methods of the present disclosure may be designed to be modular, thereby allowing them to be easily customized to meet the specific needs of a given test.

Moreover, the methods and systems of the present disclosure can be used to perform a range of mechanical tests, including angular, linear, and torsional strain testing. The methods and systems of the present disclosure may also be designed to be cost-effective and easy to use, thereby making them accessible to a wide range of users. The methods and systems of the present disclosure can also help address the lack of available and suitable equipment for testing the mechanical reliability of implantable lead systems, thereby providing flexible, customizable, and cost-effective platforms for performing these tests.

In particular embodiments, the methods and systems of the present disclosure can be used to perform a range of mechanical tests under accelerated testing conditions by adjusting various test parameters (e.g., loading rate, magnitude, and/or temperature). For instance, with reference to system 10 in FIG. 1, a test specimen 12 may be placed in a test chamber 14, which is then sealed and coupled to the chamber heating module 32. The chamber heating module 32 can then be used to heat up the content of the test chamber 14 (e.g., saline solution 15) to replicate harsh operating conditions. The combined effect of the high rates of stress and elevated temperatures serves as a catalyst to accelerate the fatigue, wear, and potential failure mechanisms that the test specimen 12 would experience over an extended period. Such conditions can enable a user to perform long-term reliability testing within a considerably reduced timeframe. In particular, such conditions can help condense what would be months or even years of real-world exposure to just a few days or weeks in the testing chamber 14.

As such, the methods and systems of the present disclosure can have numerous applications. For instance, in some embodiments, the methods and systems of the present disclosure may be used as a platform for evaluating the mechanical performance of implantable leads used in implantable electrical stimulation systems, such as for peripheral nerve interfaces, cardiac pacing devices, functional electrical stimulation devices, spinal cord stimulation devices, or combinations thereof.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein

1. A system for testing the mechanical stability of a material, said system comprising:
a chamber comprising a first end and a second end, wherein the first end and the second end are on opposite ends of the chamber;
a motion actuating unit associated with the first end of the chamber;
a first base area associated with the motion actuating unit at or near the first end of the chamber,
wherein the first base area is operational to connect to a first end of the material, and
wherein the motion actuating unit is operational to move the first base area and the immobilized material; and
a second base area associated with the second end of the chamber,
wherein the second base area is operational to connect to a second end of the material, wherein the second end of the material is on an opposite end of the first end of the material.

2. The system of claim 1, wherein the motion actuating unit comprises a motor operational for actuating motion.

3. The system of claim 2, wherein the actuated motion comprises repetitive rotational motion.

4. The system of claim 2, wherein the motion actuating unit further comprises a motion transfer assembly operational for transferring the actuated motion from the motor to the first base area for moving the first base area and the immobilized material.

5. The system of claim 1, wherein the first base area comprises a moving clamp, and wherein the second base area comprises a fixed clamp.

6. The system of claim 1, further comprising an optical monitoring system, wherein the optical monitoring system is operational to optically monitor the material associated with the system.

7. The system of claim 1, further comprising a heater associated with the chamber, wherein the heater is operational to heat the chamber.

8. The system of claim 1, further comprising an impedance measurement system, wherein the impedance measurement system is operational to measure the impedance of the material associated with the system, and wherein the impedance measurement system comprises a multimeter and a computing device.

9. The system of claim 1, wherein the chamber is in the form of a transparent cylinder.

10. The system of claim 1, wherein the system is operational for testing the mechanical stability of a plurality of materials.

11. The system of claim 10, wherein the system comprises:
a plurality of first base areas associated with the motion actuating unit, wherein each of the first base areas is operational to connect to a first end of one of the plurality of materials, and wherein the motion actuating unit is operational to move each of the first base areas; and
a plurality of second base areas associated with the second end of the chamber, wherein each of the second base areas is operational to connect to a second end of one of the plurality of materials.

12. The system of claim 11, wherein the motion actuating unit comprises a motor operational for actuating motion, and a motion transfer assembly operational for transferring the actuated motion from the motor to each of the first base areas for moving each of the first base areas and each of the immobilized materials.

13. The system of claim 12, wherein the motion transfer assembly comprises:
a first vertical arm connected to the motor;
a horizontal arm connected to the first vertical arm; and
a plurality of second vertical arms, wherein each of the plurality of second vertical arms is connected to the horizontal arm and one of the plurality of first base areas, and wherein the horizontal arm is operational to transform motion from the motor to linear motion through an adjustable tilted axis, and wherein each of the second vertical arms are operational to receive the transformed linear motion and move each of the first base areas and each of the plurality of the immobilized materials.

14. The system of claim 1, wherein the material comprises an implantable electrode.

15. The system of claim 1, wherein the tested mechanical stability is selected from the group consisting of strain, angular strain, linear strain, torsional strain, stress, torsion, mechanical fatigue, or combinations thereof.

16. A method for testing the mechanical stability of a material through the utilization of the system, wherein the system comprises:
a chamber comprising a first end and a second end, wherein the first end and the second end are on opposite ends of the chamber,
a motion actuating unit associated with the first end of the chamber,
a first base area associated with the motion actuating unit at or near the first end of the chamber, and
a second base area associated with the second end of the chamber; and
wherein the method comprises:
connecting a first end of the material to the first base area of the system,
connecting a second end of the material to the second base area of the system, wherein the second end of the material is on an opposite end of the first end of the material,
actuating the motion actuating unit, wherein the actuating moves the first base area and the immobilized material, and
measuring the mechanical stability of the material.

17. The method of claim 16, wherein the motion actuating unit comprises a motor and a motion transfer assembly, wherein the actuating results in the transfer of the actuated motion from the motor to the first base area for moving the first base area and the immobilized material.

18. The method of claim 16, further comprising a step of optically monitoring the material associated with the system, wherein the optical monitoring occurs through the utilization of an optical monitoring system associated with the system.

19. The method of claim 16, further comprising a step of heating the chamber, wherein the heating occurs through the actuation of a heater associated with the chamber.

20. The method of claim 16, wherein the testing of the mechanical stability of the material comprises measuring the impedance of the material, wherein the impedance of the material is measured by an impedance measurement system.

21. The method of claim 16, further comprising a step of filling the chamber with a liquid, wherein the filling occurs prior to measuring the mechanical stability of the material.

22. The method of claim 16, wherein the method is used to test the mechanical stability of a plurality of materials.

23. The method of claim 22, wherein the system comprises:
a plurality of first base areas associated with the motion actuating unit, and
a plurality of second base areas associated with the second end of the chamber; and wherein the method comprises:
connecting each of the first base areas to a first end of one of the plurality of materials, and
connecting each of the second base areas to a second end of one of the plurality of the materials,
actuating the motion actuating unit, wherein the actuating moves each of the first base areas and each of the immobilized materials, and
measuring the mechanical stability of each of the materials.

24. The method of claim 23, wherein the motion actuating unit comprises a motor operational for actuating motion and a motion transfer assembly operational for transferring the actuated motion from the motor to each of the first base areas for moving each of the first base areas and each of the immobilized materials, wherein the motion transfer assembly comprises:
a first vertical arm connected to the motor,
a horizontal arm connected to the first vertical arm, and
a plurality of second vertical arms, wherein each of the plurality of second vertical arms is connected to the horizontal arm and one of the plurality of first base areas; and
wherein after actuating, the horizontal arm transforms motions from the motor to linear motion through an adjustable tilted axis, and each of the second vertical arms receive the transformed linear motion and move each of the first base areas and each of the plurality of the immobilized materials.

25. The method of claim 22, wherein the material comprises an implantable electrode.

26. The method of claim 22, wherein the tested mechanical stability is selected from the group consisting of strain, angular strain, linear strain, torsional strain, stress, torsion, mechanical fatigue, or combinations thereof.
